(19) United States
(12) Patent Application Publication
(10) Pub. No.: US 2023/0258531 A1
(43) Pub. Date: Aug. 17, 2023

(54) METHOD AND SYSTEM FOR GEARBOX FAILURE DETECTION USING RADIOACTIVE COMPONENTS

(71) Applicant: Bell Textron Inc., Fort Worth, TX (US)

(72) Inventor: Eric Stephen OLSON, Fort Worth, TX (US)

(73) Assignee: Bell Textron Inc., Fort Worth, TX (US)

(21) Appl. No.: 17/671,279

(22) Filed: Feb. 14, 2022

(51) Int. Cl. G01M 13/021; G01N 33/28

(52) U.S. Cl. CPC G01M 13/021; G01N 33/2858

(57) ABSTRACT

A system includes a chip detector that includes a magnet, the chip detector operable to collect a plurality of chips in a lubricant flow. The system also includes a radiation sensor associated with the magnet and operable to detect radiation emitted by at least some of the plurality of chips collected by the magnet.

### TECHNICAL FIELD

The present disclosure relates generally to chip detectors used with gearboxes and more particularly, but not by way of limitation, to use of radioactive materials in conjunction with chip detectors commonly used to detect problems in gearboxes of rotary-wing aircraft, such as helicopters, as well as other aircraft.

### BACKGROUND

This section provides background information to facilitate a better understanding of various aspects of the disclosure. It should be understood that statements in this section of this document are to be read in this light and not as admissions of prior art.

Rotary-wing aircraft such as helicopters use lubricant-distribution systems that distribute lubricant to one or more gearboxes and other components of the rotary-wing aircraft. Typical lubricant-distribution systems include mechanisms operable to detect different types of lubricant contaminants. These mechanisms often include what are referred to as chip detectors operable to capture and to detect metallic contaminants in the lubricant being distributed. Chip detectors may be electric or magnetic and are typically operable to act on metallic components via use of one or more magnets.

Failures of components of, for example, gearboxes, are often results in pieces breaking loose from the components. These pieces are often referred to as chips, which chips may be detected by a chip detector. The chips are typically collected by a chip detector as they are distributed by the lubrication-distribution system. Chip detectors typically have a magnetic element. The magnetic element may be removed on a periodic basis from a gearbox or other component with which it is associated and inspected for chips collected thereby. In other systems, the chip detector sends a signal indicative of the presence of chips.

Typical chip detectors cannot detect non-ferrous materials such as, for example, ceramics used in hybrid bearings. This detection problem is one reason for limited use of hybrid bearings in helicopter gearboxes. In addition, typical chip detectors cannot determine the origin of chips detected. As such, false positives from chip detectors may result because the chip detector has detected foreign-object debris (“FOD”) in the gearbox such as, for example, machining shavings or shot-peen pellets, rather than chips. Also, in the case of a real positive, current chip detectors cannot determine from which component a particular chip originated.

### SUMMARY

This summary is provided to introduce a selection of concepts that are further described below in the detailed description. This summary is not necessarily intended to identify key or essential features of the claimed subject matter, nor is it intended to be used as an aid in limiting the scope of claimed subject matter.

A system includes a chip detector that includes a magnet, the chip detector operable to collect a plurality of chips in a lubricant flow. The system also includes a radiation sensor associated with the magnet and operable to detect radiation emitted by at least some of the plurality of chips collected by the magnet.

A system includes a filter operable to collect a plurality of chips in a lubricant flow and a radiation sensor associated with the filter and operable to detect radiation emitted by the at least some of the plurality of chips.

A system for detecting chips in a lubricant flow. The system includes a chip-collection apparatus selected from the group consisting of a chip detector, a filter operable to collect chips larger than 3 μm, and a screen. The system also includes a radiation sensor operable to detect radiation emitted by at least some of the collected chips. The radiation sensor is at least one of in the filter, adjacent to the screen, and adjacent to the chip detector.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion.

FIG. 1 shows illustrative radiation sensors;

FIG. 2 is an enlarged partial view of a gearbox assembly;

FIG. 3 is a partial cross-sectional view of a chip detector inserted into the gearbox of FIG. 2;

FIG. 4 is a partial cross-sectional view of a gearbox assembly equipped with a radiation sensor; and

FIG. 5 illustrates a filter assembly.

### DETAILED DESCRIPTION

Various embodiments will now be described more fully with reference to the accompanying drawings. The disclosure may, however, be embodied in many different forms and should not be construed as limited to the embodiments set forth herein.

One way faults in gearboxes are detected is through chip detectors. In some embodiments, if chips detected by the chip detector are significant enough to close a circuit of the chip detector, the chip detector is removed so the chips can be analyzed. However, oftentimes, the chips detected by the chip detector are from components that are not pertinent to potential failure of an important component or are from FOD. In addition, in many cases, it is not possible to detect consistently and reliably the component from which the chips originated.

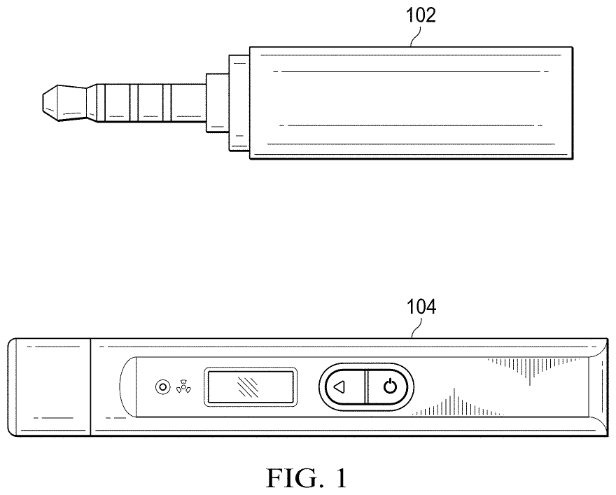

FIG. 1 shows illustrative radiation sensors 102 and 104. The radiation sensor 102 and the radiation sensor 104 are relatively small and readily available. The radiation sensor 102 has a mass of 6 g and can detect 0.1-200 μSv/hr, while the radiation sensor 104 has a mass of 30 g and can detect 0.01-999 μSv/hr. Thus, relatively small amounts of radiation that would not be harmful to humans are detectable by commercially available radiation sensors.

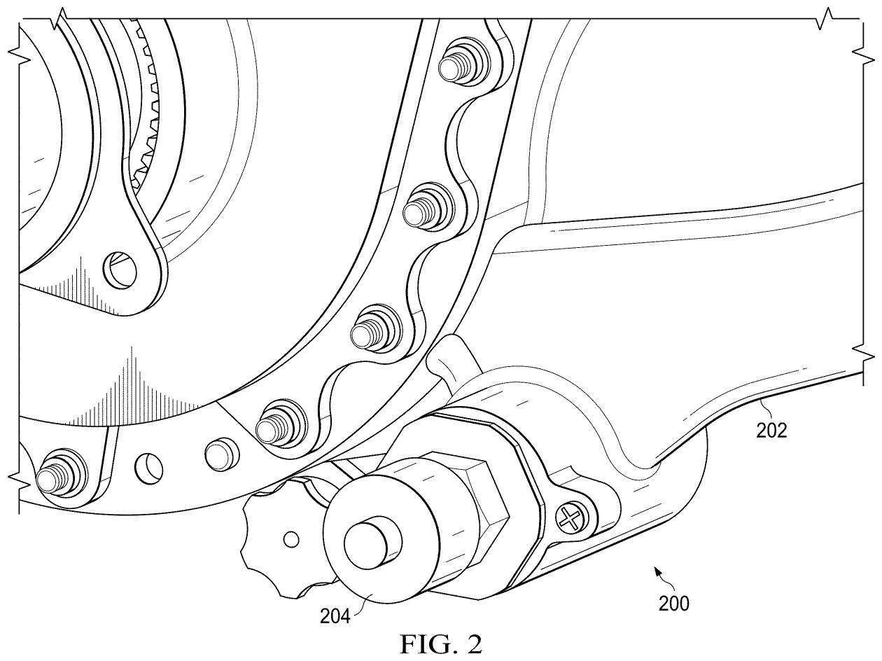

FIG. 2 is an enlarged partial view of a gearbox assembly 200. The gearbox assembly 200 includes a gearbox 202 and a chip detector 204. The chip detector 204 may be unscrewed and thereby separated from the gearbox 202 for analysis of chips captured by the chip detector 204.

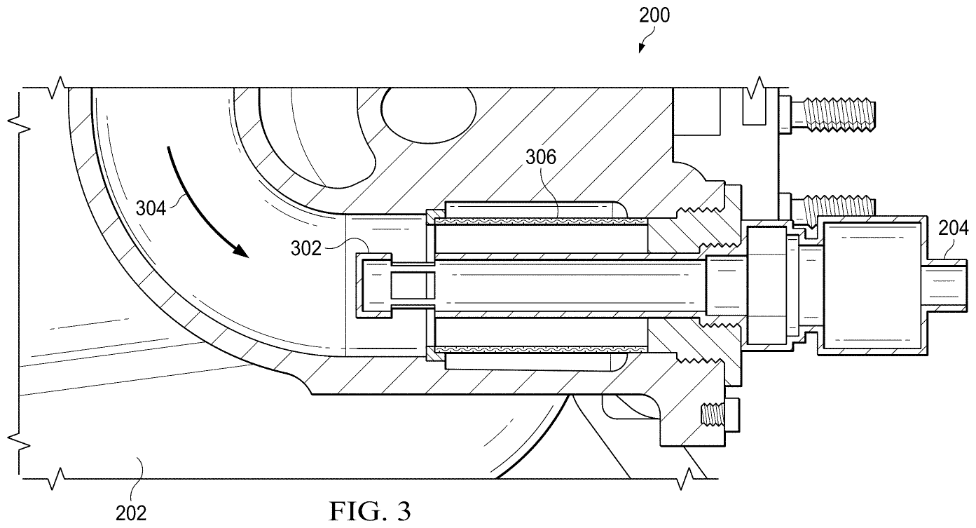

FIG. 3 is a partial cross-sectional view of the chip detector 204 inserted into the gearbox 202. The chip detector 204 includes a magnet 302 that captures ferrous chips in oil flow illustrated by arrow 304 through the gearbox 202 as the chips pass by the chip detector 204. The gearbox 202 also includes a filter operable to capture larger chips in the oil flow 304 and referred to as a screen, the screen being designated by reference numeral 306. In some embodiments, if chips or FOD detected by the chip detector 204 are significant enough to close a circuit of the chip detector 204, the chip detector 204 may be removed so the chips can be analyzed. However, oftentimes, the chips detected by the chip detector 204 are from unimportant components. In other cases, the chip detector 204 detects FOD, which by definition is unimportant. In addition, it is not possible via use of the chip detector 204 to determine the component from which detected chips originated. In a typical embodiment, the oil flow 304 proceeds as follows: 1) sump of the gearbox 202; 2) the chip detector 204; 3) the screen 306; 4) a pump; 5) a filter; 6) a heat exchanger; and back to the gearbox 202.

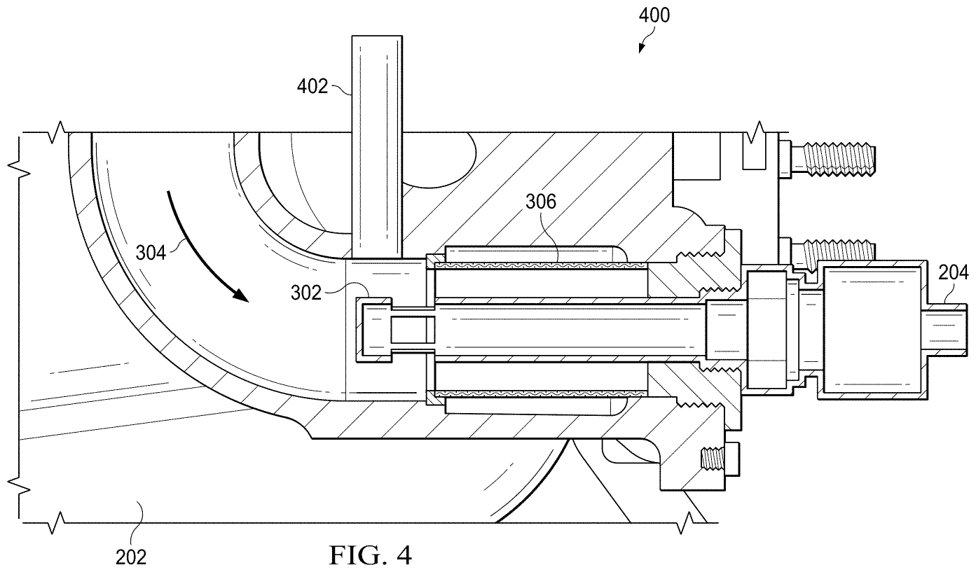

FIG. 4 is a partial cross-sectional view of a gearbox assembly 400 equipped with a radiation sensor 402. The gearbox assembly 400 includes the gearbox 202 and the chip detector 204. The chip detector 204 includes the magnet 302 that captures magnetic chips in the oil flow 304 as the chips pass by the chip detector 204. The gearbox 202 also includes the screen 306 that captures larger chips in the oil flow 304. The radiation sensor 402 is shown associated with the chip detector 204 adjacent to the magnet 302. In similar fashion to FIG. 3, in a typical embodiment, the oil flow 304 proceeds as follows: 1) sump of the gearbox 202; 2) the chip detector 204; 3) the screen 306; 4) a pump; 5) a filter; 6) a heat exchanger; and back to the gearbox 202.

In other embodiments, a modification of the gearbox assembly 400 may be made such that the gearbox assembly 400 does not include the chip detector 204. In such an embodiment, a radiation sensor such as, for example, the radiation sensor 402, can be positioned adjacent to the screen 306 and detect radioactive isotopes associated with chips caught by the screen 306. In some embodiments, a first radiation sensor may be positioned adjacent to the chip detector 204 and a second radiation sensor positioned adjacent to the screen 306.

In a typical embodiment, various components of the gearbox 202 are intentionally produced with radioactive isotopes in order that the radiation sensor 402 can identify the particular component from which chips originated. In similar fashion, chips from unimportant components, as well as FOD, can, in some embodiments, be ignored.

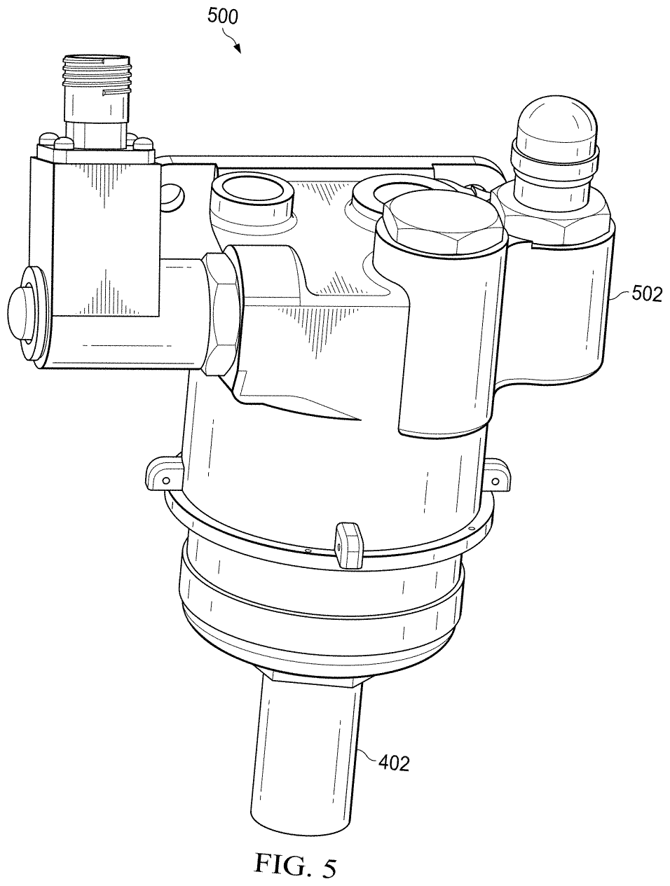

FIG. 5 illustrates a filter assembly 500. The filter assembly 500 includes a filter 502 and the radiation sensor 402 inserted into the filter 502. In some embodiments, the filter 502 captures chips and the radiation sensor 402 is utilized to detect radioactive isotopes in the captured chips.

In this way, a particular component from which the chips originated can be reliably identified. Typical filters such as the filter 502 are capable of filtering chips greater than 3 μm. In similar fashion to the above, in a system that employs the filter assembly 500, oil flow proceeds as follows: 1) a gearbox sump; 2) a screen to filter larger chips; 3) a pump; 4) the filter 502 and the radiation sensor 402; 6) a heat exchanger; and 7) back to the gearbox.

Therefore, one or more radiation sensors may be used to detect radioactive isotopes of chips collected by one or more of a screen, a filter, and a chip detector. In some embodiments, a signal indicative of detected radioactive isotopes at one or more of the screen, the filter, and the chip detector is transmitted to a pilot or to a flight control computer. Various different radioactive isotopes that emit different types of radiation (e.g., alpha, beta, gamma) can be utilized with different components in order to distinguish between the components when the chips have been detected by the radiation sensor. In such cases, chips from various different components would have a unique radiation signature.

Radioactive isotopes can be utilized with both metals and non-metals; as such, chips from non-ferrous and non-metallic components that would not be detectable by a magnetic chip detector could be identified by the radiation sensor when the chips from the non-ferrous and non-metallic components are encountered by the radiation sensor. One example of a non-metallic and non-ferrous component is silicon nitride used in rolling elements of hybrid bearings.

The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” “generally,” and “about” may be substituted with “within 10% of” what is specified.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments are possible in which these tasks are performed by a different entity.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A system comprising:
a chip detector comprising a magnet, the chip detector operable to collect a plurality of chips in a lubricant flow; and
a radiation sensor associated with the magnet and operable to detect radiation emitted by at least some of the plurality of chips collected by the magnet.

2. The system of claim 1, wherein the detected radiation has a pre-defined radiation signature.

3. The system of claim 2, wherein the pre-defined radiation signature is unique to a particular component.

4. The system of claim 1, wherein the radiation comprises first radiation with a first radiation signature and second radiation with a second radiation signature.

5. The system of claim 4, wherein:
the first radiation signature is unique to a first component; and
the second radiation signature is unique to a second component.

6. The system of claim 1, wherein the emitted radiation is indicative of an important component.

7. The system of claim 6, wherein chips that do not emit radiation indicate an unimportant component.

8. The system of claim 1, wherein at least some of the plurality of chips originate from a component produced so as to generate a pre-defined radiation signature.

9. A system comprising:
a filter operable to collect a plurality of chips in a lubricant flow; and
a radiation sensor associated with the filter and operable to detect radiation emitted by the at least some of the plurality of chips.

10. The system of claim 9, wherein the radiation sensor is operable to detect a radiation signature of the emitted radiation.

11. The system of claim 10, wherein the radiation signature is unique to a particular component.

12. The system of claim 9, wherein the filter is operable to collect chips greater than 3 μm.

13. The system of claim 9, wherein:
the radiation sensor is operable to detect a plurality of radiation signature; and
each of the plurality of radiation signatures is unique to a particular component.

14. The system of claim 9, wherein the at least some of the plurality of chips are non-ferrous.

15. The system of claim 13, wherein the at least some of the plurality of chips are non-metallic.

16. The system of claim 9, wherein at least some of the plurality of chips originate from a component produced so as to generate a pre-defined radiation signature.

17. A system for detecting chips in a lubricant flow, the system comprising:
a chip-collection apparatus selected from the group consisting of:
a chip detector;
a filter operable to collect chips larger than 3 μm; and
a screen;
a radiation sensor operable to detect radiation emitted by at least some of the collected chips; and
wherein the radiation sensor is at least one of:
in the filter;
adjacent to the screen; and
adjacent to the chip detector.

18. The system claim 17, wherein:
the radiation emitted has a pre-defined radiation signature indicative of a particular component;
the particular component is produced so as to generate the pre-defined radiation signature; and
chips of the collected chips that do not emit a pre-defined radiation signature originate from an unimportant component.

19. The system of claim 17, wherein at least some of the chips are non-ferrous and non-metallic.

20. The system of claim 17, wherein:
the emitted radiation comprises at least two different radiation signatures;
a first radiation signature of the at least two different radiation signatures is indicative of a first component; and
a second radiation signature of the at least two different radiation signatures is indicative of a second component.
